(19) United States
(12) Patent Application Publication
(10) Pub. No.: US 2005/0113915 A1
(43) Pub. Date: May 26, 2005

(54) Jet skin mesher

(76) Inventor: Quint A. Marcaletti, New Philadelphia, OH (US)

Correspondence Address: ZIMMER TECHNOLOGY - ROBERTS;INDIANO, VAUGHN & ROBERTS, 1 NORTH PENNSYLVANIA AVENUE #850, INDIANAPOLIS, IN, 46204, US

(21) Appl. No.: 10/721,856

(22) Filed: Nov. 25, 2003

(51) Int. Cl. A61F 2/10; B26D 7/14

(52) U.S. Cl. 623/15.12; 83/18; 83/22; 83/175; 83/177

(57) ABSTRACT

An apparatus for jet cutting skin into a mesh pattern is provided. The apparatus includes a nozzle, a table spaced apart from the nozzle, and a removable skin carrier positioned on the table. The table and the nozzle are automatically relatively movable according to the mesh pattern.

### FIELD OF THE INVENTION

The present invention relates generally to the field of skin grafting, and, more particularly, to skin meshers.

### BACKGROUND

A first degree or superficial burn heals naturally through the body's ability to replace damaged skin cells. Deep second and full thickness burns require skin graft surgery for quick healing and minimal scarring. Skin grafting involves the surgical removal of burn injured tissue and cleaning of the wound (“excision”), choosing healthy (“donor”) skin to be used for covering the excised area, and removing the graft from the donor site (“harvesting”) with an instrument (“dermatome”) that is similar to an electric shaver. The dermatome typically shaves a piece of skin about 10/1000 of an inch thick from the donor site. The donor skin is surgically secured over the excised area so that it can heal.¹
¹ http://www.cooltheburn.com/learn/sugery/grafts.html, Oct. 9, 2002.

Very large areas of open wounds are difficult to cover because there might not be enough unburned donor skin available. For such large wounds it may be desirable to enlarge donor skin to cover larger body surface areas. Meshing is a way to enlarge donor skin. Meshing involves making small slits in the donor skin, which allows expansion similar to fish netting. In a meshed skin graft, the skin from the donor site may be stretched to allow it to cover a larger area. After application of a meshed graft, healing occurs as the spaces between the mesh (“intricities/intricity”) fill in with new epithelial skin growth. Meshing also allows blood and body fluids to drain from under the graft, reducing the risks of graft loss.¹
¹ http://www.cooltheburn.com/learn/sugery/grafts.html, Oct. 9, 2002.

In general, industrial water jet cutting is known. For example, U.S. Pat. No. 6,125,729 issued to Mirabello on Oct. 3, 2000 (“Mirabello”), which is incorporated herein, shows a multiple head cutting unit for cutting leather, animal skin and synthetic materials utilized in the footwear and leather goods sector with a water jet. However, conventional skin meshing devices use metal blades for cutting slits into the donor skin. In some designs the size and spacing between the slits is limited by the physical constraints of machining and controlling the metal blades. Additionally, some blades generate undesirable amounts of heat as they work and, in some systems the skin tends to adhere to the blades. Moreover, metal blades can eventually dull from repeated use and cleaning.

### SUMMARY OF THE INVENTION

The present invention provides an apparatus for jet cutting skin into a mesh pattern. The apparatus includes a nozzle, a table spaced apart from the nozzle, and a removable skin carrier positioned on the table. The table and the nozzle are automatically relatively movable according to the mesh pattern.

In an alternative embodiment, the present invention provides an apparatus for jet cutting skin into a mesh pattern. The apparatus includes a means for automatically jet cutting the skin into the mesh pattern, and a means, coupled to the jet cutting means, for tensioning the skin.

In another alternative embodiment, the present invention provides a method for jet cutting skin. The method includes the steps of automatically jet cutting the skin into a selectable mesh pattern, and automatically tensioning the skin concurrently with the jet cutting step.

In another alternative embodiment, the present invention provides an article of manufacture including jet meshed human skin.

The above-noted features and advantages of the present invention, as well as additional features and advantages, will be readily apparent to those skilled in the art upon reference to the following detailed description and the accompanying drawings.

### BRIEF DESCRIPTION OF THE DRAWINGS

FIG. 1 shows a schematic block diagram of a piece of human donor skin in an exemplary jet skin mesher according to the present invention; and

FIG. 2 shows a piece of jet meshed human skin according to the present invention.

### DETAILED DESCRIPTION OF EXEMPLARY EMBODIMENT(S)

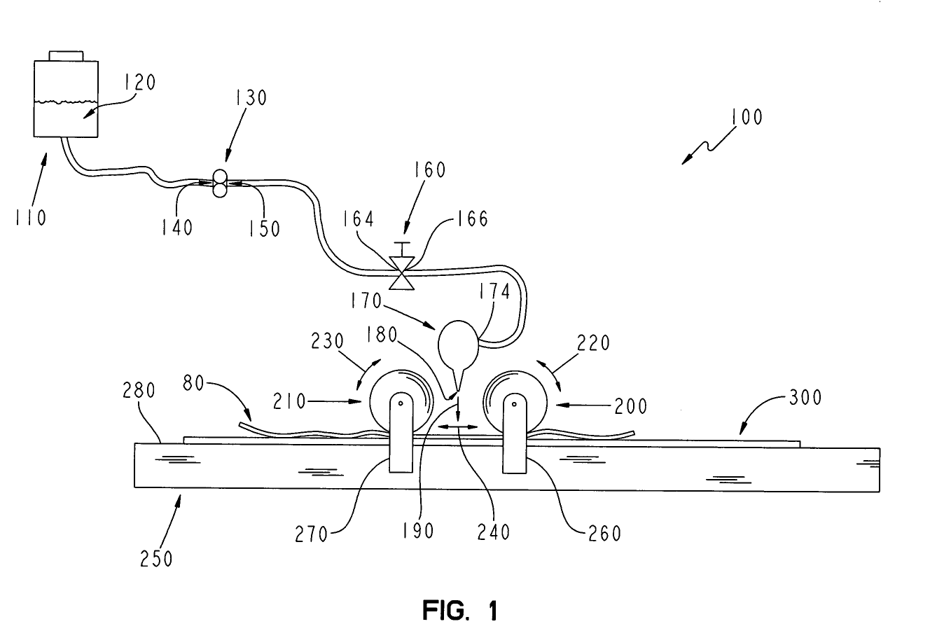

FIG. 1 shows a schematic block diagram of a piece of human donor skin 80 in an exemplary jet skin mesher 100 according to the present invention. Mesher 100 includes a source 110. In the exemplary embodiment, source 110 is a suitable reservoir containing a suitable biocompatible saline solution 120.

Mesher 100 further includes a pump 130. Pump 130 includes an inlet 140 that is hydraulically coupled to source 110, and further includes an outlet 150. Pump 130 is configured to receive solution 120 through inlet 140 and force solution 120 through outlet 150 at a suitably high pressure.

Mesher 100 also includes an electrically controlled valve 160. Valve 160 includes an inlet 164 that is hydraulically coupled to outlet 150 of pump 130, and further includes an outlet 166. Additionally, valve 160 is electrically coupled to a suitable controller (not shown). Valve 160 is configured to open and close fluid communication between inlet 164 and outlet 166 in response to suitable signals from the controller. In the exemplary embodiment, the controller includes suitable software (not shown) to suitably time the opening and closing of valve 160 and to control other operations of mesher 100 as discussed below.

Mesher 100 further includes a jet nozzle 170. Nozzle 170 includes an inlet 174 that is hydraulically coupled to outlet 166 of valve 160; and nozzle 170 defines an orifice 180. Nozzle 170 is configured to emit a focused jet stream, spray, or burst of solution 120 (see directional arrow 190) from orifice 180 to suitably cut skin 80 with the jet stream when valve 160 is opened.

Mesher 100 also includes a roller 200 and an opposing roller 210. Roller 200 and roller 210 include respective electric motors (not shown) that are electrically coupled to the afore-mentioned controller, suitably configured to receive suitable signals from the controller, and suitably configured to turn (see directional arrows 220, 230) roller 200 and roller 210 in opposite directions (i.e., clockwise and counterclockwise, or vice-versa) or in the same direction (but at slightly different speeds) in response to the signals such that the skin 80 is suitably tensioned (see directional arrows 240) so that it remains generally flat as it is cut by the jet stream emitted from orifice 180 of nozzle 170.

Mesher 100 also includes an x-y table 250, a bracket 260 that couples table 250 to roller 200, and a bracket 270 that couples table 250 to roller 210. Table 250 includes suitable electric motors (not shown) that are electrically coupled to the afore-mentioned controller and configured to move the table generally perpendicularly to the jet stream emitted from orifice 180 (i.e., generally perpendicularly to directional arrow 190) in response to suitable signals from the controller. Table 250 includes an upper, generally planar surface 280 that faces towards roller 200 and roller 210.

Additionally, mesher 100 includes a generally planar disposable sterile skin tray or carrier 300. Carrier 300 is removably secured to surface 280 of table 250.

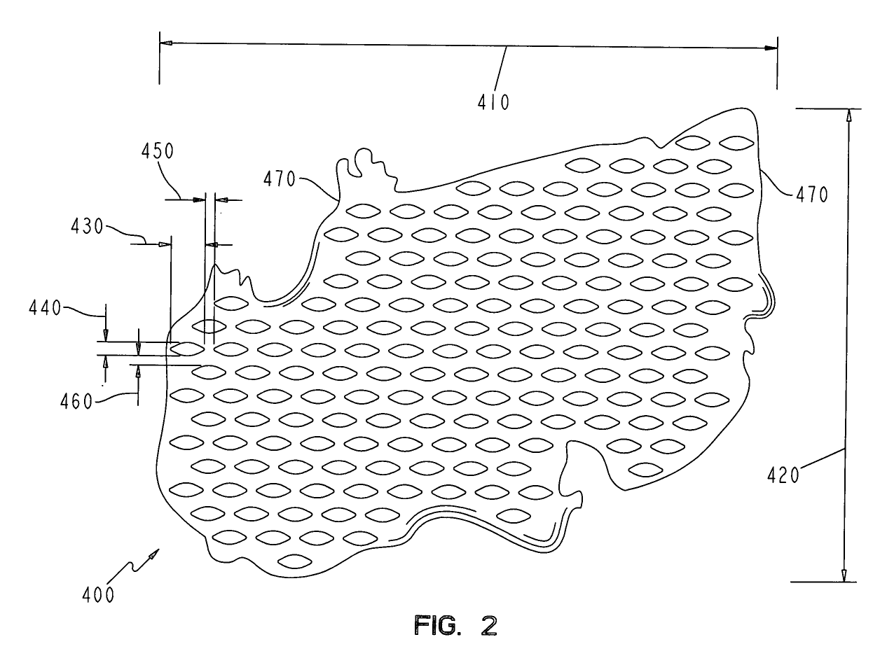

In operation of mesher 100, a user removes carrier 300, places skin 80 onto carrier 300, and then secures carrier 300 (with skin 80 thereon) to surface 280 of table 250. The user selects or defines a desired mesh pattern and a perimeteral shape (see FIG. 2) through operation of the afore-mentioned controller according to the suitable software program stored and running therein. The controller automatically causes table 250 to move, valve 160 to open and close, and roller 200 and roller 230 to turn, in synchronism, to form the desired mesh pattern (including the desired intricity widths, intricity lengths, and desired spacing between intricities) in skin 80, and to cut skin 80 into the desired perimeteral shape (see FIG. 2). After skin 80 is meshed and shaped, the user removes carrier 300 (with skin 80 thereon) and uses carrier 300 to transport skin 80 to a storage site for preservation or to an operating site for use in more immediate skin grafting procedures.

FIG. 2 shows a piece of jet meshed human skin 400 according to the present invention. Skin 400 has been stretched by hand or any other suitable manner after being meshed and perimeterally shaped by a jet skin mesher (for example, exemplary jet skin mesher 100) according to the present invention. In the exemplary embodiment, skin 400 has a major length 410, a major width 420, a generally uniform intricity or intricitial length 430, a generally uniform intricity or intricitial width 440, a generally uniform longitudinal intricity or intricitial spacing 450, a generally uniform lateral intricity or intricitial spacing 460, and a perimeteral outline or shape 470. Length 430, width 440, spacing 450, and spacing 460 define an overall “mesh pattern” for skin 400. While it should be appreciated that the mesh pattern of FIG. 2 is merely exemplary, and that mesher 100 (see FIG. 1) may be configured to facilitate practically any number of desired mesh patterns, it is noted that in the preferred embodiment spacing 460 is around 0.025 inches after jet cutting skin 80 and prior to stretching of skin 80 (see FIG. 1) to produce skin 400 (see FIG. 2). Conventional bladed meshers cut skin with a distance of about 0.050 inches between the blades. Decreasing the distance between the cuts allows a mesh of a given ratio to have a smaller distance for skin to heal across (“epethelialize”), thereby reducing healing time and scarring. This should also reduce the risk of infection. Because there are no blades to heat or stick to the skin, jet cutting allows this spacing to be significantly reduced over the prior art.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. An apparatus for jet cutting skin into a mesh pattern, the apparatus comprising:
a nozzle;
a table spaced apart from the nozzle; and
a removable skin carrier positioned on the table;
wherein the table and the nozzle are automatically relatively movable according to the mesh pattern.

2. The apparatus of claim 1, further comprising a skin tensioner coupled the table.

3. The apparatus of claim 2, wherein the skin tensioner further includes a first skin roller spaced apart from the removable skin carrier.

4. The apparatus of claim 3, wherein the skin tensioner further includes a second skin roller spaced apart from the removable skin carrier and spaced apart from the first skin roller.

5. An apparatus for jet cutting skin into a mesh pattern, the apparatus comprising:
a means for automatically jet cutting the skin into the mesh pattern; and
a means, coupled to the jet cutting means, for tensioning the skin.

6. The apparatus of claim 5, wherein the tensioning means includes a removable means for carrying the skin.

7. A method for jet cutting skin, the method comprising the steps of:
automatically jet cutting the skin into a selectable mesh pattern; and
automatically tensioning the skin concurrently with the jet cutting step.

8. The method of claim 7, wherein the jet cutting step includes making a first cut in the skin, the jet cutting step includes making a second cut in the skin, and the first cut is spaced apart from the second cut by about 0.025 inches.

9. The method of claim 8, wherein the step of jet cutting the skin into the mesh pattern includes jet cutting human skin.

10. The method of claim 9, further comprising the step of automatically jet cutting the skin into a predetermined perimeteral shape.

11. The method of claim 7, wherein the step of jet cutting the skin into the mesh pattern includes jet cutting human skin.

12. The method of claim 7, wherein the step of jet cutting the skin into the mesh pattern includes jetting a biocompatible saline solution.

13. An article of manufacture, comprising:
jet meshed human skin.

14. The article of claim 13, wherein the skin defines a first intricity, the skin defines a second intricity, and the first intricity is spaced apart from the second intricity by about 0.025 inches.

15. The article of claim 13, wherein the skin is stretched.
